(19) United States
(12) Patent Application Publication
(10) Pub. No.: US 2021/0022786 A1
(43) Pub. Date: Jan. 28, 2021

(54) METHODS AND DEVICES FOR DELIVERING MEDICAL MATERIAL

(71) Applicant: NORDSON CORPORATION, WESTLAKE, OH (US)

(72) Inventors: Zachary Rzeszutek, St. Paul, MN (US); Mark Stevenson, St. Paul, MN (US); Patrick M. Parkinson, Boulder, CO (US); Katie M. Baldwin, Boulder, CO (US); Tyler C. Grubb, Boulder, CO (US); Clayton D. Cole, Boulder, CO (US); Diane C. Keyser, Boulder, CO (US); David S. Hazlitt, Boulder, CO (US)

(21) Appl. No.: 16/640,808

(22) PCT Filed: Aug. 24, 2018
(86) PCT No.: PCT/US2018/047915

§ 371 Date: Feb. 21, 2020

Related U.S. Application Data
(60) Provisional application No. 62/549,539, filed on Aug. 24, 2017.

(51) Int. Cl. A61B 17/88; A61B 90/00

(52) U.S. Cl. CPC A61B 17/8816; A61B 17/8822; A61B 2090/3966; A61B 90/39; A61B 17/8833

(57) ABSTRACT

Methods and apparatuses for preparing and delivering medical material are disclosed. A material delivery apparatus includes a sheath that defines a lumen therethrough, a trough having an inner surface that defines a material receptacle, the trough being configured to slidably pass into the lumen, and a stylet configured to slidably pass into the material receptacle. The shape of the stylet complements the inner surface of the trough and a portion of the lumen when the trough is in the lumen of the sheath.

### CROSS REFERENCE TO RELATED APPLICATIONS

This application claims the benefit of U.S. Provisional Application No. 62/549,539, filed Aug. 24, 2017, the entirety of which is incorporated herein for any and all purposes.

### TECHNICAL FIELD

This disclosure is directed to preparing and dispensing medical material and, more particularly, an improved delivery apparatus incorporating a trough and a keyed stylet.

### BACKGROUND

Bone degradation is a common side effect of various infirmities, injuries and the general aging process. Additionally, bone voids are created during certain surgical procedures that require the re-growth of bone. Surgical procedures that seek to replace, fuse, or alleviate the ailments associated with bone damage or voids are complex and often difficult to perform. A bone graft is a surgical transplant of bone tissue that comes from the patient's body, a donor, or another species to fill the voids or to alleviate the ailments of bone degradation. Bone grafts can also be useful for growing new bone tissue around an implanted device, such as spinal fusion or a knee replacement.

Materials used in bone grafting are often rough and heterogeneous, making them difficult to prepare, mix and apply. Known methods include preparing and mixing the necessary materials in a mixing bowl and then using a tool, such as a spatula, to transfer the mixed material into a dispenser, which can then be used to apply the mixed material at the desired location. However, it is tedious and time-consuming work to gather the mixed material from the mixing bowl using a spatula, transfer the mixed material to a funnel, and then to transfer the mixed material from the funnel into the patient at the desired application spot. Moreover, the amount of time required to repeatedly transfer the mixed material, as well as the wasted material resulting from the repeated transfers, increases the cost of a bone grafting procedure.

Therefore, there is a need for improved devices and methods for preparing and transferring a medical material, such as a bone grafting material, to a patient.

### SUMMARY

Devices and methods for delivering medical material are disclosed. According to an embodiment, a material delivery apparatus includes a sheath that defines a lumen therethrough, a trough having an inner surface that defines a material receptacle, the trough being configured to slidably pass into the lumen, and a stylet configured to slidably pass into the material receptacle. The shape of the stylet complements the inner surface of the trough and a portion of the lumen when the trough is in the lumen of the sheath.

According to another embodiment, a material delivery apparatus for delivering bone graft material to a surgical site includes a sheath that defines a lumen therethrough, a trough configured to slidably pass into the lumen and having an inner surface that defines a material receptacle, and a stylet configured to slidably pass into the material receptacle. The stylet has a cross-sectional shape that complements the cross-sectional shape of a void defined between the inner surface of the trough and the sheath when the trough is within the lumen of the sheath.

According to another embodiment, a method of loading bone graft material into a material delivery apparatus having a sheath, a trough, and a keyed stylet is disclosed. The bone graft material is introduced into a material receptacle defined by an inner surface of the trough. The trough is then slidably positioned into a lumen defined by the sheath. The keyed stylet is then slid into the entirety of a void defined by the sheath and the trough.

According to another embodiment, a stylet for insertion into a sheath includes a first region with a first circular cross section having a first diameter, a second region with a second cross section having a second diameter, and a distal end configured to contact a material. The second diameter is different from the first diameter.

According to another embodiment, a method of loading bone graft material into a material delivery apparatus having a sheath defining a material receptacle therein between a proximal end and a distal end is disclosed. The method sheath is provided in an open configuration, the bone graft material is introduced into the sheath, and the sheath is transitioned from the open configuration to the closed configuration.

According to another embodiment, a method of loading bone graft material into a material delivery apparatus having a sheath, a stylet, and a funnel having an inlet and an outlet opposite the inlet is disclosed. The funnel is provided with the sheath disposed adjacent to the outlet of the funnel. The bone graft material is introduced into the inlet of the funnel, and the stylet is translated in the funnel between the inlet and the outlet such that the bone graft material is moved from the funnel through the outlet into the sheath.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific methods, devices, and systems disclosed. In addition, the drawings are not necessarily drawn to scale. In the drawings:

FIG. 1 shows an isometric view of a delivery system according to one embodiment;

FIG. 2 shows an exploded view of the delivery system shown in FIG. 1;

FIG. 3 shows an isometric view of a trough;

FIG. 4 shows an isometric view of a stylet;

FIG. 5 shows the stylet partially slid in the trough;

FIG. 6 shows an isometric end view of the trough inside a sheath;

FIG. 7 shows an isometric top view of a handle of the sheath;

FIG. 8 shows an isometric bottom view of the handle shown in FIG. 7;

FIG. 9 shows an isometric view of a container housing the sheath, trough, and stylet;

FIG. 10 shows an isometric view of the trough with a tray;

FIG. 11 shows an isometric view of another tray with a trough;

FIG. 12 shows an isometric view of another tray with two troughs;

FIG. 13 shows an isometric view of a packing tool having a scraper and a tamping head;

FIG. 14 shows an isometric view of a tray with a trough and a tamping head;

FIG. 15 shows a front perspective view of the tray, trough, and tamping head of FIG. 14;

FIG. 16 shows an isometric view of another tray;

FIG. 17 shows an isometric view of the tray of FIG. 16 adjacent a trough;

FIG. 18 shows an isometric view of another tray having a wall;

FIG. 19 shows a front cross-sectional view of a delivery system according to another embodiment;

FIG. 20 shows an isometric view of a sheath with a hinge in a closed position;

FIG. 21 shows an isometric view of the sheath of FIG. 20 in an open position;

FIG. 22 shows an isometric view of a sheath of another embodiment having two portions;

FIG. 23 shows a front perspective view of the sheath of FIG. 22 in an open position;

FIG. 24 shows a front perspective view of the sheath of FIGS. 22 and 23 in a closed position;

FIG. 25 shows a front perspective view of a sheath according to yet another embodiment;

FIG. 26 shows an isometric view of a portion of a stylet according to an embodiment;

FIG. 27 shows a side cross-sectional view of the stylet of FIG. 26 inside a sheath;

FIG. 28 shows a side cross-sectional view of a stylet according to another embodiment and having a blade; and

FIG. 29 shows a front perspective view of the stylet of FIG. 28.

### DETAILED DESCRIPTION OF ILLUSTRATIVE EMBODIMENTS

A delivery system is used to prepare and deliver medical materials. The delivery system may be disposable and designed for single use, or may be reused. The delivery system may be used to prepare and deliver a single material, or may be used to prepare, mix, and deliver multiple materials. A trough having a longitudinal opening along its length first receives the medical material. The trough with the material is then inserted into a sheath having a lumen extending therethrough. Finally, a stylet having the cross-sectional shape complementing the cross-sectional shape of a void defined between the inner surface of the trough and the sheath is inserted through the lumen to push the material out of the system. This system allows for easier loading of the material into the sheath with minimal material waste during dispensing.

Certain terminology is used in the description for convenience only and is not limiting. The words “proximal” and “distal” generally refer to positions or directions toward and away from, respectively, an individual using the delivery system. The words “axial”, “vertical”, “transverse”, “left”, “right”, “above,” and “below” designate directions in the drawings to which reference is made. The term “substantially” is intended to mean considerable in extent or largely but not necessarily wholly that which is specified. The terminology includes the above listed words, derivatives thereof, and words of similar import.

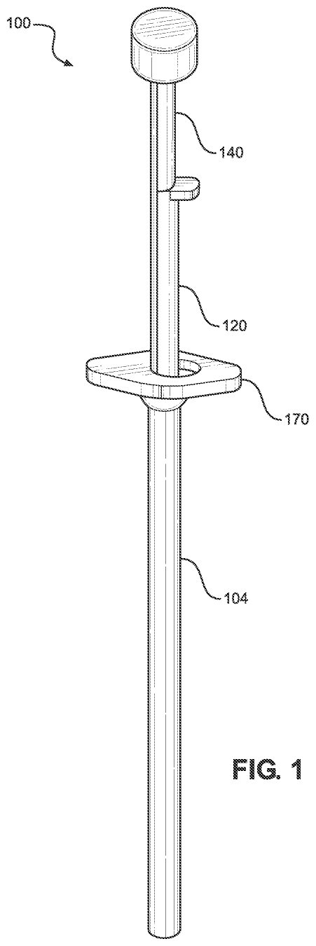

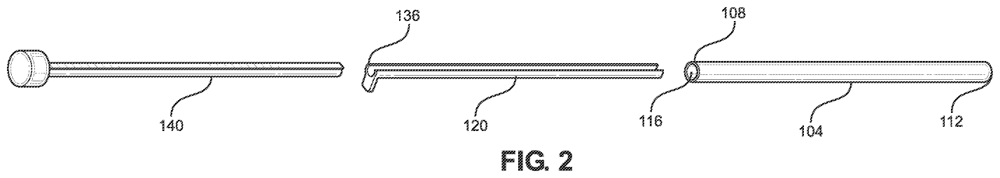

Referring to FIGS. 1 and 2, a delivery system 100 may include a sheath 104, a trough 120, and a stylet 140. The sheath 104 defines a lumen 116 that extends through the length of the sheath 104 from its proximal end 108 to its distal end 112. The sheath 104 has an opening at its proximal end 108 and another opening at its distal end 112 to deliver the material. The sheath 104 may be substantially cylindrical and uniform in size. In some embodiments, the sheath 104 may taper, for example from its proximal end 108 to its distal end 112. The sheath 104 may be made of a material suitable for surgical use. The sheath 104 may include plastics, for example polyethylene terephthalate (PET), polypropylene, acrylonitrile butadiene styrene (ABS), polyethylene terephthalate glycol-modified (PETG), a combination thereof, or another suitable polymer. In some embodiments, the sheath 104 may include metal, for example stainless steel that may be sanitized for repeated use.

The sheath 104 may include markings (not shown) along its length. The markings may include measurements, material information, dates, or other information. The markings may be printed, may be protrusions, or may be notches. In some embodiments, the markings may be radiopaque such that they are readily observable by a practitioner under X-ray or other radiation. As such, a practitioner may visualize under radiation the amount of material inside the lumen 116 and the position of the delivery system 100 within a patient. In some embodiments, the sheath 104 may be at least partially translucent, so that a practitioner may visualize the amount of material in the lumen under visible light.

The lumen 116 that extends through the sheath 104 may be substantially cylindrical. In some embodiments, the lumen 116 may be defined by the sheath 104 to be a different shape, for example prismatic, and the disclosure is not limited to a particular shape of the lumen 116. The lumen 116 is sized and dimensioned such that it can removably receive the trough 120, the stylet 140, or the combination of the trough 120 and the stylet 140.

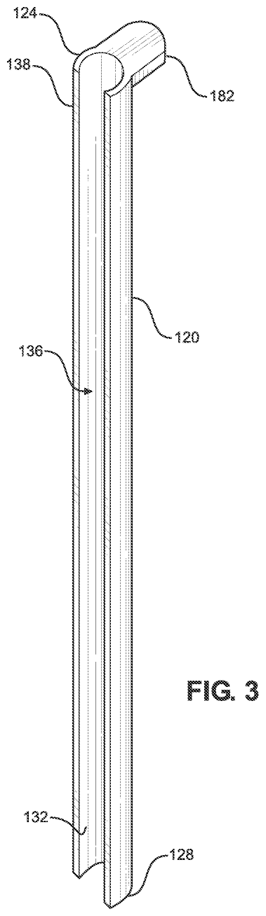

Referring to FIGS. 2 and 3, the trough 120 has a proximal end 124, a distal end 128 opposite the proximal end 124, and an inner surface 132 that defines a material receptacle 136. Material may be introduced into the material receptacle 136 such that it contacts the inner surface 132. The trough 120 may have a mouth 138 extending along at least a portion of the trough 120. The mouth 138 may be defined by a break in the inner surface 132, and the combination of the inner surface 132 and the mouth 138 may define the material receptacle 136. As shown in FIG. 3, the mouth 138 may extend along the entire length of the trough 120. In some embodiments, the mouth 138 may extend through only a segment of the trough 120.

The trough 120 slides into the lumen 116. The trough 120 has a length substantially the same as the length of the sheath 104. In some embodiments, the trough 120 may be longer or in the alternative, shorter than the sheath 104. When the trough 120 is inside the lumen 116, at least a portion of the material may be transferred out of the material receptacle 136 and out of the sheath 104, for example to a desired delivery site. When the trough 120 is within the sheath 104, the material receptacle 136 may be defined between the trough 120 and the lumen 116.

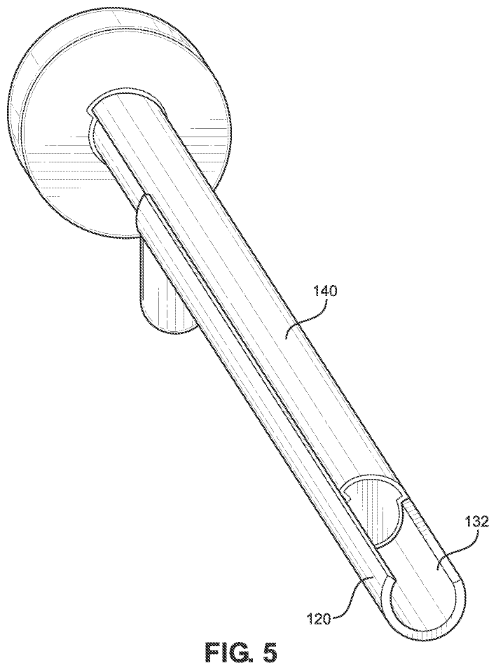

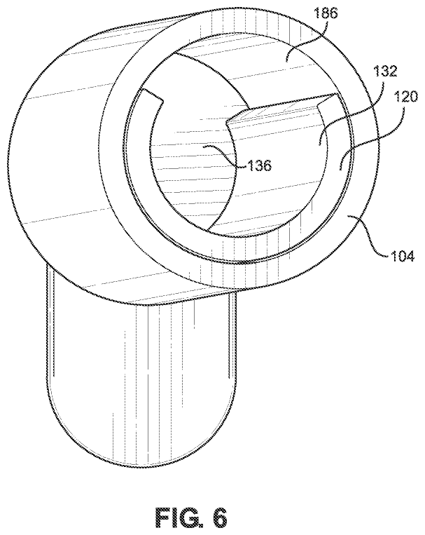

When the trough 120 without material is inserted into the lumen 116, the trough 120 and the sheath 104 define a void 186 (shown in FIG. 6) between the mouth 138 and the lumen 116. When the trough 120 includes some material, the void 186 is partially filled with material. The remainder of the void 186 provides buffer space inside the lumen 116 when material is present. This may be advantageous because the buffer space allows for material to move around more freely than in the absence of such a void, as the density of the material can cause the material to be stuck and clog the lumen 116. As such, the buffer space may decrease clogging or poor mixing of the material and may increase ventilation. Increased ventilation within the sheath 104 may obviate the requirement of a ventilation hole within the sheath 104, which is necessitated by some existing technology

The trough 120 may include plastics, for example polyethylene terephthalate (PET), polypropylene, acrylonitrile butadiene styrene (ABS), polyethylene terephthalate glycol-modified (PETG), polycarbonate (PC), high-density polyethylene (HDPE), nylon, a combination thereof, or another suitable polymer. In some embodiments, the trough 120 may include metal, for example stainless steel that may be sterilized for repeated use. The trough 120 may include markings (not shown) to indicate measurements, material information, or part reference numbers. The markings may be printed, may be protrusions, or may be notches. In some embodiments, the markings may be radiopaque such that they are readily observable by a practitioner under X-ray or other radiation. As such, a practitioner may visualize the amount of material inside the trough 120 under radiation.

Introducing material into the trough 120 and then sliding the trough 120 into the sheath 104 improves the efficiency of placing the material into the sheath in comparison to directly loading the material into the sheath 104. In other words, it can be simpler to load the trough 120 through the mouth 138 rather than pushing the material into the proximal end 108 of the sheath 104.

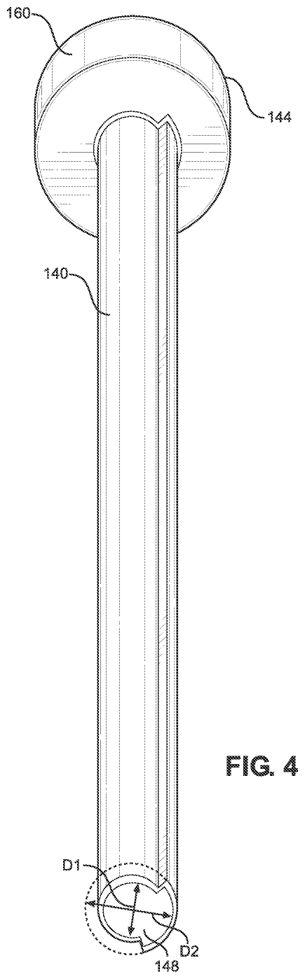

Referring to FIGS. 4-6, the stylet 140 is configured to move all or a portion of the material in the void 186 out of the lumen 116. The stylet 140 includes a handle 160 disposed on the stylet's proximal end 144. The stylet 140 is inserted into the lumen 116 at the proximal end 108 of the sheath 104 and slides through the lumen 116 to push material out of the distal end 112 of the sheath 104. The stylet 140 is dimensioned to be about as long as the trough 120, the sheath 104, or both the trough 120 and the sheath 104. In alternative embodiments, the stylet 140 may be either longer or shorter than the trough 120 and/or sheath 104. Several advantages exist for different stylet lengths. A stylet 140 longer than the sheath 104, for example, may be useful to ensure that most or all of the material inside the void 186 is pushed out of the sheath 104. A stylet 140 that is shorter than the sheath may be advantageous to ensure that the distal end of the stylet 140 does not extend beyond the distal end 112 of the sheath 104 and contact a dispensing surface, which may damage a biological site or tissue.

In some embodiments, the stylet 140 is shaped and dimensioned such that the stylet 140 complements the shape and dimensions of the void 186. The cross section of the void 186 corresponds to a region between the lumen 116 and the inner surface 132 of the trough 120. The stylet 140 may be keyed to the shape of the void 186, such that the stylet 140 may pass through the lumen 116 in only one orientation and cannot be rotated within the lumen 116. The keyed stylet 140 is advantageous because it increases the likelihood of removing most or all material from the lumen relative to a typical round stylet and reduces the risk of using an incorrect stylet.

As shown in FIG. 4, the stylet 140 includes more than one diameter. The stylet 140 has a first cylindrical portion with a first diameter D1 and a second cylindrical portion with a second diameter D2 that is larger than the first diameter D1. Referring to FIG. 5, the first portion having the first diameter D1 of stylet 140 complements the inner diameter of the trough 120. The second portion having the second diameter D2 of stylet 140 complements the inner diameter of the lumen 116. As such, the stylet 140 pushes material out of the entirety of the void 186 through the distal end 112 of the sheath 104.

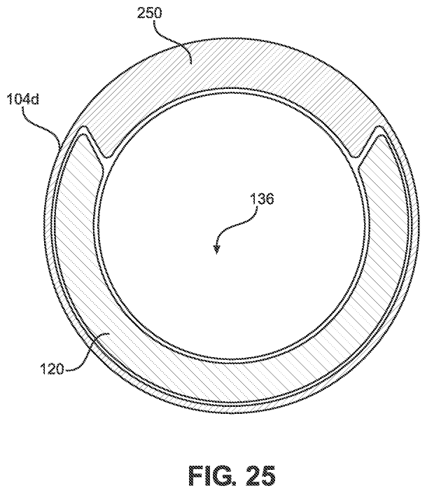

In some embodiments, the sheath may be keyed to have a specific shape instead of the stylet 140. It will be understood that either the sheath, the stylet, or both the sheath and stylet can be keyed to have one or more shapes that complement each other and act to prevent insertion and/or movement of the stylet 140 in an undesired orientation through the sheath 104 (see, for example, FIG. 25, showing a keyed sheath 104d having a keyed portion 250 extending towards the material receptacle 136).

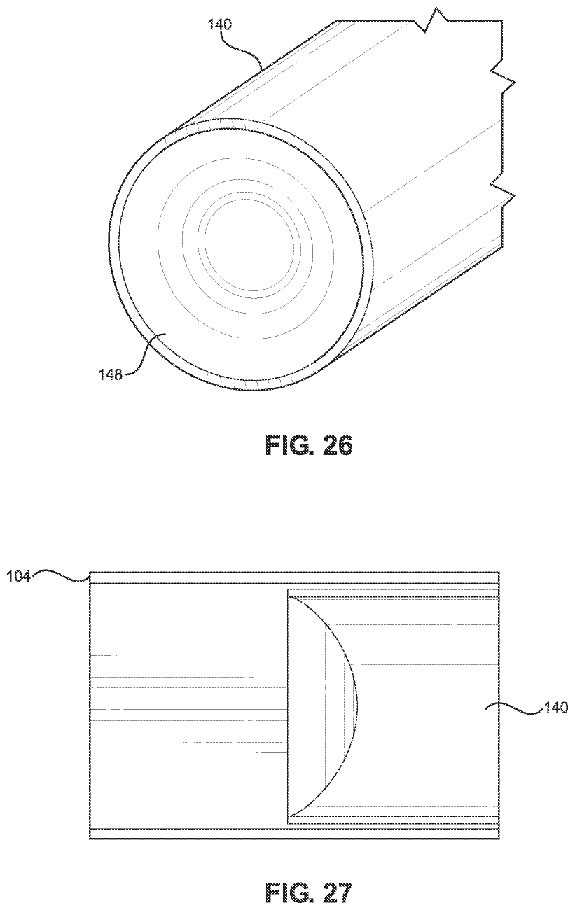

The stylet 140 may be sloped or tapered at its distal end 148. The slope or taper may be angled such that when the distal end 148 contacts the material, the material is pushed out of the trough 120 and into the remainder of the void 186. In another embodiment, the distal end 148 of the stylet 140 may be concave, such that the material is pushed towards the centerline of the stylet 140 and/or the lumen 116 (see, for example, FIGS. 26 and 27). In an alternative embodiment, the distal end 148 may be convex, such that the material is pushed away from the centerline of the stylet 140 and/or lumen 116. Various advantages may exist for different stylet shapes, such as increased precision when dispensing material, better spreading of the material within the lumen 116, and reduced clogging of the material inside the sheath 104.

The stylet 140 may include plastics, for example polyethylene terephthalate (PET), polypropylene, acrylonitrile butadiene styrene (ABS), polyethylene terephthalate glycol-modified (PETG), polycarbonate (PC), high-density polyethylene (HDPE), nylon, a combination thereof, or another suitable polymer. In some embodiments, the stylet 140 may include metal, for example stainless steel that may be sterilized for repeated use. The stylet 140 may include markings (not shown) to indicate measurements, material information, or part reference numbers. The markings may be printed, may be protrusions, or may be notches. In some embodiments, the markings may be radiopaque such that they are readily observable by a practitioner under X-ray or other radiation. As such, the practitioner may see how far the stylet 140 has travelled within the lumen 116, thus indicating how much material has been moved out of the sheath 104.

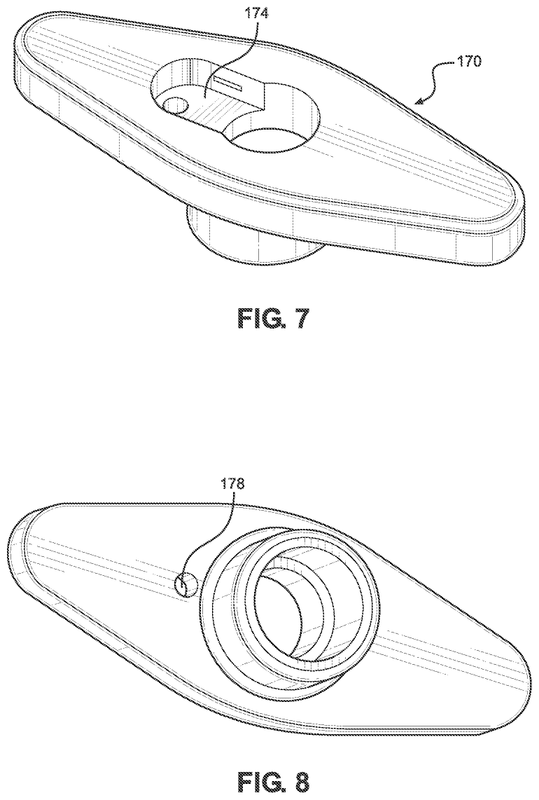

Additional components may be included in the delivery system. FIGS. 7 and 8 illustrate a grip 170 that may be attached to the proximal end 108 of the sheath 104. The grip 170 may be designed to retain the trough 120 from inadvertent or premature removal from the sheath 104. Referring still to FIGS. 7 and 8, the grip 170 may include a locking recess 174 configured to engage with a corresponding locking projection 182 of the trough 120. It will be understood, however, that this disclosure is not limited to the locking mechanism described above. The grip 170 allows a practitioner of the delivery system 100 to hold the sheath 104 rigidly while loading the trough into the delivery system 100 and pushing the stylet 140 through the lumen 116.

In some embodiments, the grip 170 may also include an unlocking aperture 178 extending through the grip 170. When the locking projection 182 of the trough 120 is engaged with the locking recess 174 of the grip 170, an unlocking tool (not shown) may be inserted through the unlocking aperture 178 to disengage the locking projection 182 from the locking recess 174. The unlocking aperture 178 is dimensioned such that one or more specific tools can fit through the unlocking aperture 178 to decrease the likelihood of accidentally disengaging the trough 120 from the sheath 104.

The delivery system 100 may be a part of a delivery kit. The delivery kit may include multiple delivery systems 100. The kit may include delivery systems for use with different materials, for example with materials of varying viscosity or material composition. Each delivery system 100 may include components described throughout this disclosure. The components may have different dimensions and shapes. In some embodiments, the kit may include a plurality of stylets 140, where each stylet has different dimensions, for example, length of the stylet, cross-sectional or keyed shape, and shape of the distal end 148.

The kit may include a plurality of troughs 120. In some embodiments, each of the troughs 120 may be dimensioned different from the other troughs. Differences may include shapes and dimensions of the mouth 138, the length of the trough 120, and the size of the material receptacle 136. One or more of these parameters may also define the size and shape of the void 186 between the lumen 116 and the material receptacle 136.

The kit may include a plurality of sheaths 104. Different sheaths may be configured for use with different materials or different applications. Sheaths 104 may vary in shape and dimensions and may be configured to engage with one or more of the components of the delivery system described herein.

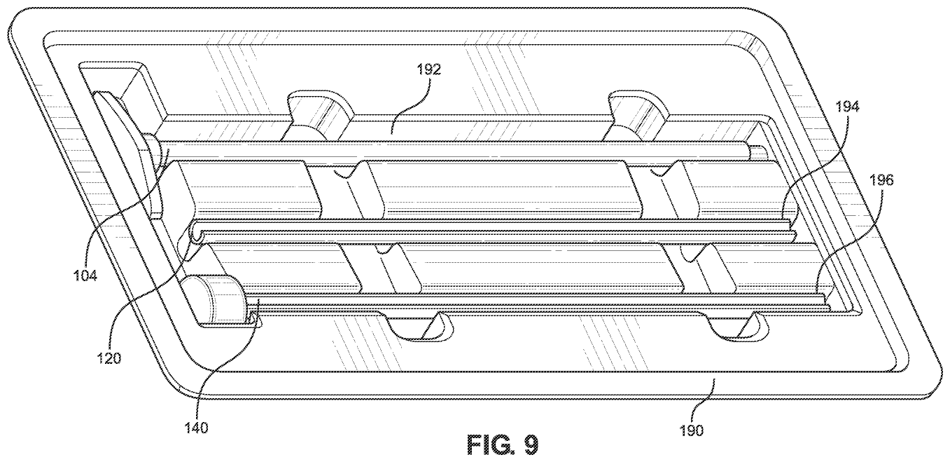

In some embodiments, the kit may also include additional components that may be used with the delivery system. In some embodiments, the kit may include a container 190 configured to house one or more components of the delivery system 100. Referring to FIG. 9, the container 190 may have a sheath receptacle 192, a trough receptacle 194, and a stylet receptacle 196. The container 190 removably houses the individual components within their respective receptacles and prevents them from being damaged or lost during shipment and material preparation.

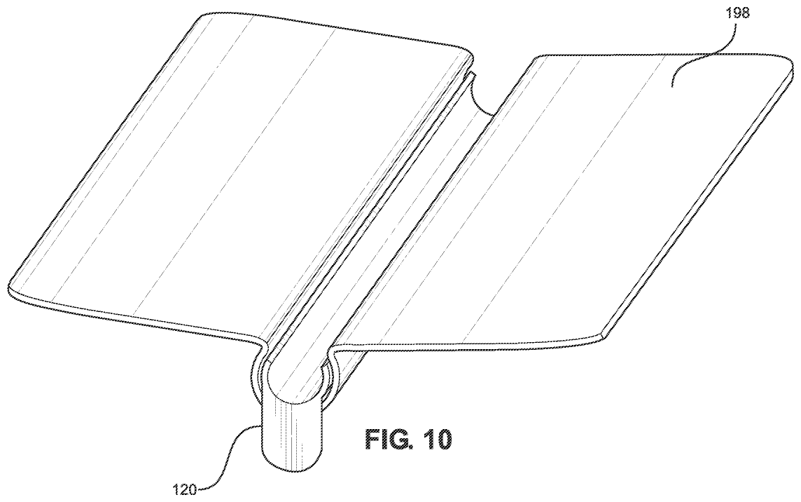

In some embodiments, the trough receptacle 194 is further configured to hold the trough 120 stationary as material is introduced into the trough 120. Referring to FIG. 10, a tray 198 may be configured to receive material to be moved into the trough 120. The tray 198 provides a larger surface area on which to deposit, mix, measure, or otherwise prepare material before introducing it into the material receptacle 136 of the trough 120. The tray 198 may be sloped in the direction of the trough 120 to ease transfer of material into the material receptacle 136. In some embodiments, the tray 198 may include a surface coating configured to decrease friction between the material and the tray 198. When the trough 120 is sufficiently filled with material, it is removed from the container 190 and from the tray 198. Referring to FIG. 11, for example, a tray 198a may be shaped such that material can be readily mixed therein and moved into the trough 120. In some aspects, the tray 198 may be part of the packaging for the included components for sales, transportation, or storage. This can provide a native environment for the individual components to be secured within the tray 198 during storage or shipment, for example, and will serve to reduce unpacking times and the required amount of packaging material.

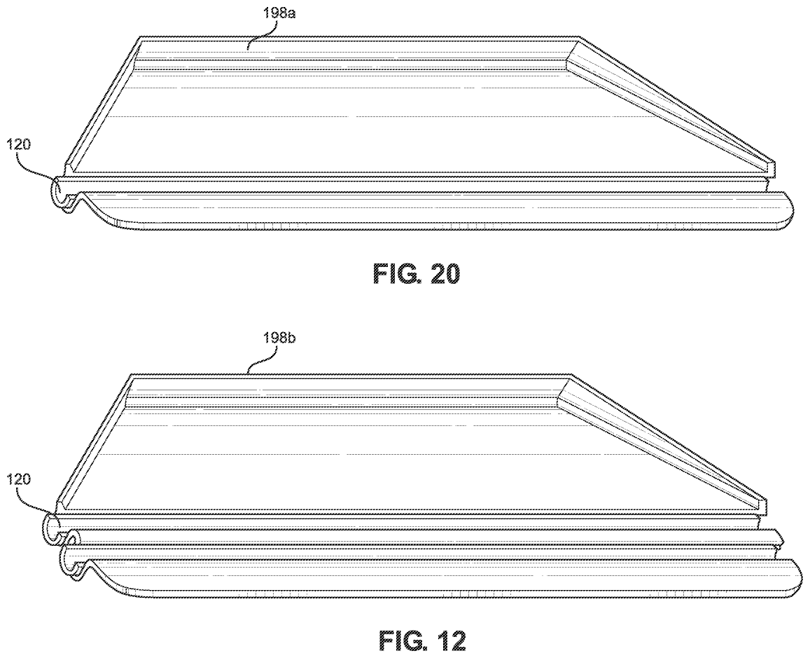

Depending on the use and the anticipated need of the material, multiple troughs 120 may be positioned to receive the mixed materials. Referring to FIG. 12, for example, a tray 198b may be configured to hold two troughs 120 and to introduce material into one or both of them as needed.

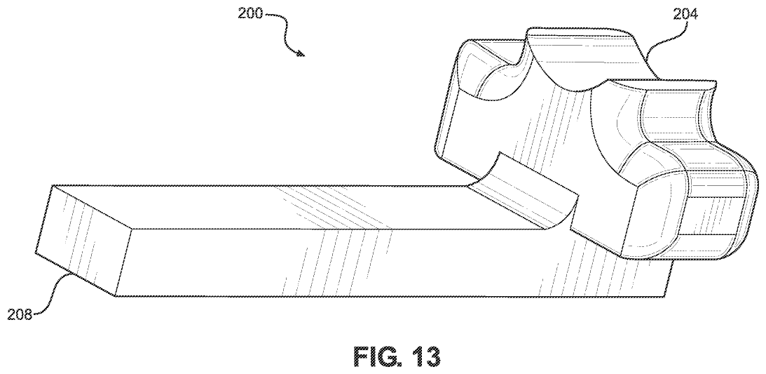

The kit may further include one or more tools for introducing the material into the trough 120 or the sheath 104. Such tools may include, but are not limited to, scoops, spatulas, funnels, bottles, pipettes, and piping bags. In some embodiments, one or more of the above tools may be used to apportion, mix, and move material on the tray 198 and into the trough 120. In some embodiments, a packing tool 200 may be used to move the material into the trough 120 or the sheath 104 and to position the material such that it can be dispensed. Referring to FIG. 13, the packing tool 200 may include a tamping head 204 and a scraper 208. The scraper 208 may be used to direct the material toward the desired trough 120 or sheath 104. Such a tool allows for collection of more material from the tray 198 and minimizes the amount of left-over unused material, which decreases waste products and minimizes material costs.

The tray 198 may also include a hinge (not shown), such that a first portion of the tray 198 receives a trough 120, while a second, hinged portion of the tray 198 is configured to rotate along the hinge towards or away from the trough 120. Such an arrangement can be used to form the packaging for the trough 120. In some aspects, the second portion of the tray 198 may be used to tamp the material introduced into the trough 120, similar to the mechanism described by the tamping head 204 of the packing tool 200.

Referring to FIGS. 14 and 15, the tamping head 204 may be used to pack the material into the trough 120 or sheath 104. This allows for fewer voids in the material and results in a more consistent dispensing of the product. Furthermore, the tamping head 204 may be contoured to facilitate formation of a desired material shape. As seen in FIGS. 14 and 15, for example, the tamping head 204 may have a curved shape, such that the tamped material in the material receptacle 136 defined between the trough and the tamping head 204 is formed to be substantially cylindrical. It will be understood, however, that the tamping head 204 (as well as the trough 120) may be contoured differently to define a different shape of the material receptacle 136 and the material disposed therein.

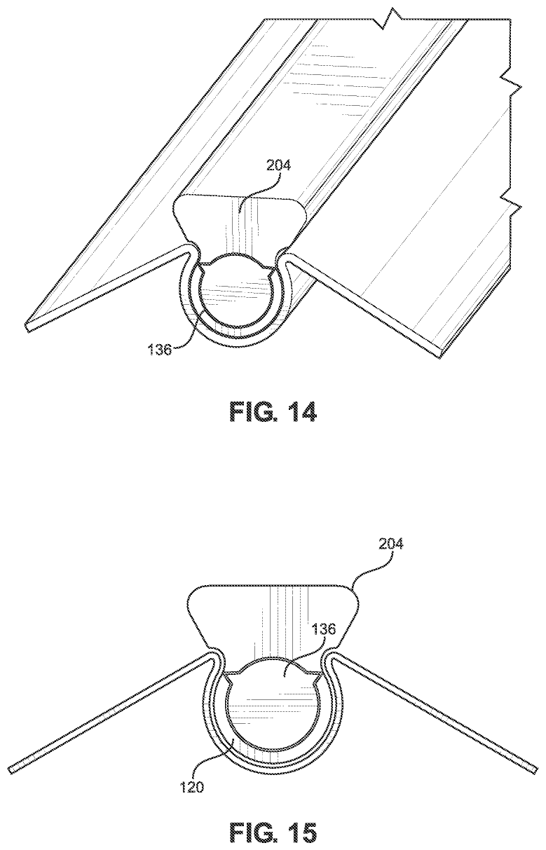

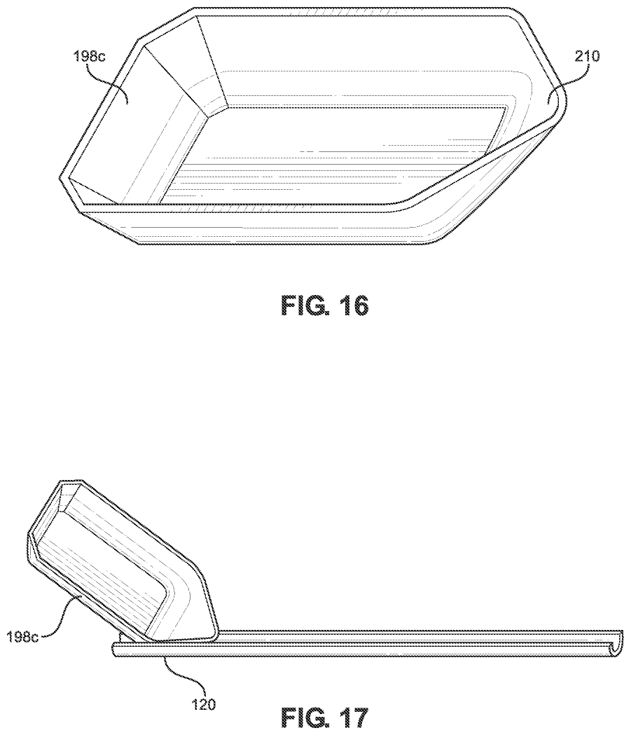

Depending on the consistency of the material, it may be advantageous to pour the prepared material into a trough or sheath from which it will be dispensed. As shown in FIGS. 16 and 17, a tray 198c may be used to prepare material therein and then to pour the material into the trough 120. In some embodiments, the tray 198c may have a spout 210 to facilitate the pouring action and to better direct the material into the trough 120. It will be understood that a tool (for example, the packing tool 200) may be used to aid in pouring the material from the tray 198c.

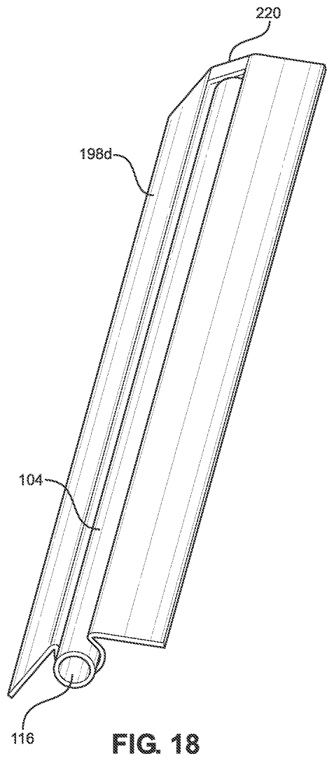

In some embodiments, the material may be moved into a sheath 104 directly without first being introduced into a trough 120. Referring to FIG. 18, for example, a tray 198d may be configured to receive the material and then to slidably receive the sheath 104. The sheath 104 may be moved along one or more surfaces of the tray 198d such that the material is received from the tray 198d into the lumen 116 of the sheath 104. In some embodiments, the tray 198d may further include a wall 220 configured to prevent the material from leaving the tray 198d and to facilitate the movement of the material into the sheath 104. In operation, the sheath 104 is moved along a surface of the tray 198d containing the material such that the material is moved from the tray 198d into the lumen 116 of the sheath 104. The tray 198d may have a shape that complements the shape of the sheath 104. Alternatively, instead of moving the sheath 104 along the tray 198d, the tray 198d may be moved relative to the stationary sheath 104.

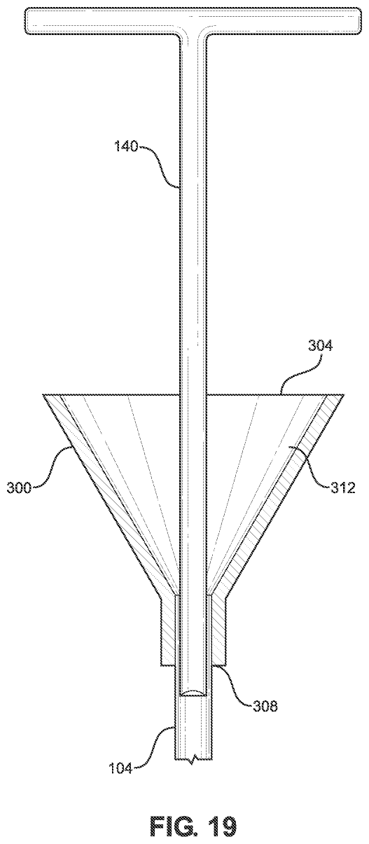

In another embodiment, a funnel 300 may be used to prepare the material and to deliver it into the sheath 104 for dispensing. Referring to FIG. 19, the funnel 300 includes an inlet 304, into which material is introduced, and an outlet 308, from which material is moved into the sheath 104, disposed opposite the inlet 304. Material may be prepared inside the funnel 300 on the inner surface 312. To facilitate movement of the material from the funnel 300 into the sheath 104, a stylet 140 may be introduced to move from the inlet 304 towards and at least partly through the outlet 308 to displace the material inside the funnel 300 and to push it into the sheath 104. The funnel 300 and stylet 140 may be utilized to prepare and move the material into the sheath 104 for future use, or the movement of the stylet 140 through the funnel 300 and the sheath 104 may be used to dispense the material.

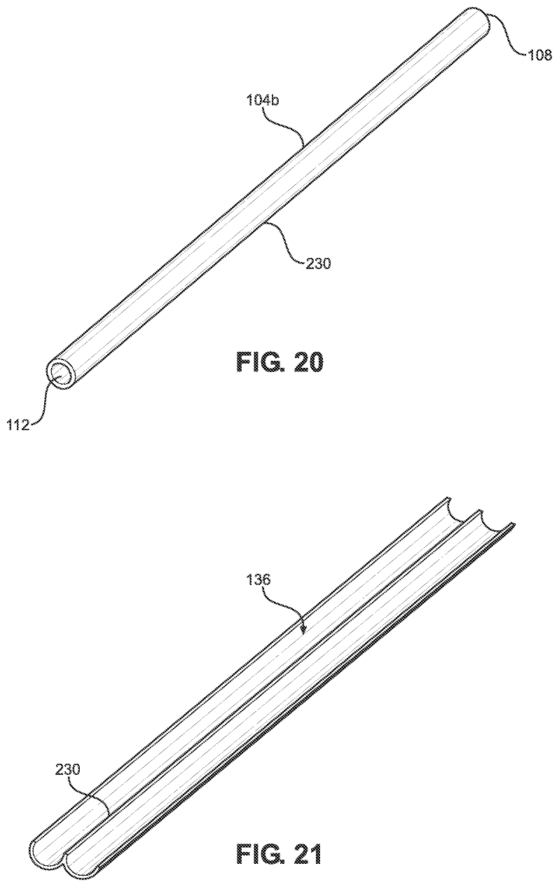

Referring to FIGS. 20 and 21, a sheath 104b may include a hinge 230 along at least a portion of the length of the sheath 104b. The hinge 230 may be a living hinge that is an integral part of the unitary sheath 104b. Alternatively, the hinge 230 may be a separate component that is attached to the sheath 104b. As shown in FIGS. 20 and 21, the hinge 230 may extend along the entire length of the sheath 104b. It will be understood, however, that the hinge 230 may extend for less than the entire length of the sheath 104b, and that a plurality of hinges 230 may be used.

The hinge 230 allows the sheath 104b to be opened and closed such that the material receptacle 136 is exposed along part of all of the length of the sheath 104b. This allows the material to be introduced directly into the sheath 104b without using a trough 120. After the material is deposited into the material receptacle 136 when the sheath 104b is in the open configuration (shown in FIG. 21), a portion of the sheath 104b can be rotated along the hinge 230 into a closed configuration (shown in FIG. 20), in which the material inside the material receptacle 136 is configured to be dispensed from the distal end 112 of the sheath 104b.

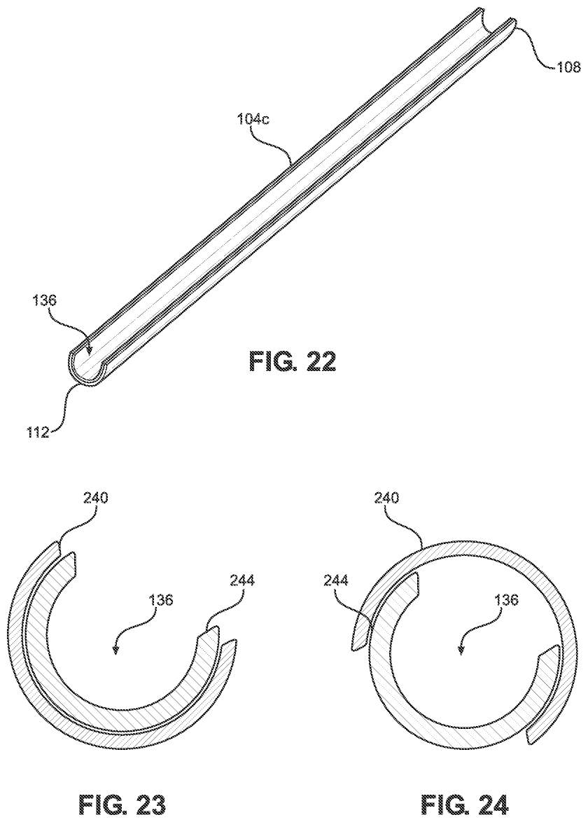

In an alternative embodiment, a sheath 104c may include a first, outer portion 240 and a second, inner portion 244 disposed adjacent to the outer portion 240 as depicted in FIGS. 22-24. The inner portion 244 and the outer portion 240 are configured to slidably rotate relative to each other, such that when the outer portion 240 is disposed opposite the inner portion 244, the sheath 104c defines the material receptacle 136 between the outer portion 240 and the inner portion 244.

The sheath 104c includes an open configuration (shown in FIG. 23), in which the material receptacle 136 is open along the length of the sheath 104c, and the inner portion 244 is disposed between the outer portion 240 and the material receptacle 136. When in the open configuration, material may be introduced into the material receptacle 136 without the use of a separate trough 120 due to the configuration of the inner portion 244 acting as a trough. After the material is loaded, the sheath 104c may be transitioned to a closed configuration (shown in FIG. 24), in which the material receptacle 136 is defined between the outer portion 240 and the inner portion 244.

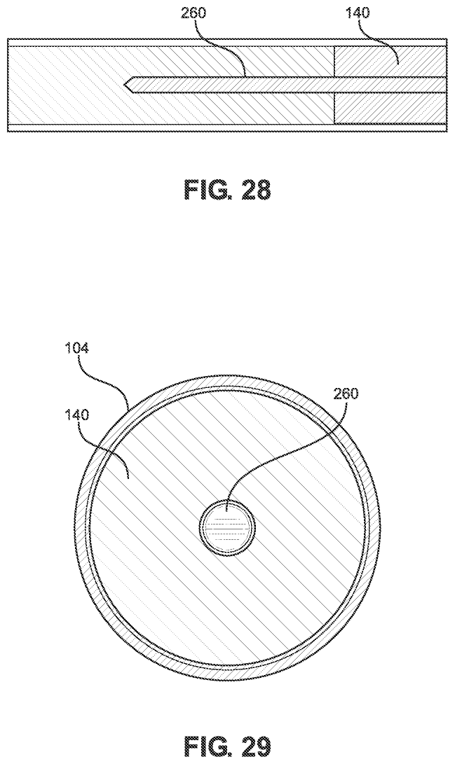

In some scenarios, it may be advantageous to agitate or stir the material after it has been introduced into the trough 120 and/or sheath 104 to prevent the material from forming large clusters, to break up any formed chunks, or to clear jams caused by larger particles. According to some embodiments, a tool may be used to disturb the material. Referring to FIGS. 28 and 29, for example, a movable blade 260 may be disposed within the sheath 104. The blade 260 may be affixed to the stylet 140 such that it moves with the stylet 140, or it may move freely and separately from the stylet 140. As shown in FIGS. 28 and 29, the blade 260 may extend through the center of the stylet 140. It will, however, be understood that the blade 260 may be disposed at a different portion of the stylet 140 or on a different component entirely. For example, the blade 260 may be disposed at the distal end 148 of the stylet and may be adjacent to the interior walls of the lumen 116 of the sheath 104 or to the inner surface 132 of the trough 120. While FIGS. 28 and 29 depict a single blade 260, a plurality of blades 260 may be utilized.

The additional components described herein may include plastics, for example polyethylene terephthalate (PET), polypropylene, acrylonitrile butadiene styrene (ABS), polyethylene terephthalate glycol-modified (PETG), polycarbonate (PC), high-density polyethylene (HDPE), nylon, a combination thereof, or another suitable polymer, as well as metal, for example stainless steel. Additionally, one or more coatings may be used on any of the disclosed components to improve reception of material, proper mixing and preparation of the material, and transfer or dispensing of the material. For example, a coating may be applied to the sheath, trough, tray, and/or stylet to decrease the relative coefficient of friction between the surface of each component and the material. This would ease moving the material without having the material catch on or stick to surfaces. Similarly, one or more coatings may be applied to the components to decrease friction between adjacent components and to improve relative movement, for example, by electropolishing or by applying a lubricious coating to the sheath and/or the trough to decrease friction when moving the trough relative to the sheath. It will be understood that various coating materials may be used, and that the separate components may be manufactured out of suitable low-friction materials instead of receiving a separate coating.

Additional tools for mixing or dispensing the material may be used with one or more of the components described throughout this application. For example, a dispensing handle (not shown) may be configured to receive the sheath and the stylet and to progress the stylet through the sheath. The handle may include a motor or a manual control mechanism, such as a lever or button. In some embodiments, the handle may include a ratcheting mechanism configured to advance the stylet a predetermined distance to correspond to a desired amount of material dispensed. Such a mechanism would allow for more precise and accurate dispensing.

In the present disclosure, the singular forms “a,” “an,” and “the” include the plural reference and reference to a particular numerical value includes at least that particular value, unless the context clearly indicates otherwise. Thus, for example, a reference to “a material” is a reference to at least one of such materials and equivalents thereof known to those skilled in the art, and so forth. It is to be understood that this disclosure is not limited to the specific devices, methods, conditions or parameters described or shown herein, and that the terminology used herein is for the purpose of describing particular embodiments by way of example only and is not intended to be limiting of any subject matter claimed.

1. A material delivery apparatus, comprising:
a sheath that defines a lumen therethrough;
a trough having an inner surface that defines a material receptacle, the trough being configured to slidably pass into the lumen; and
a stylet configured to slidably pass into the material receptacle,
wherein the shape of the stylet complements the inner surface of the trough and a portion of the lumen when the trough is in the lumen of the sheath.

2. The material delivery apparatus of claim 1, wherein the stylet is shaped such that the stylet passes into the lumen and the material receptacle in only one orientation.

3. The material delivery apparatus of claim 1, wherein the trough further includes a locking element configured to releasably secure the trough to the sheath when the trough is in the lumen, such that the trough is prevented from rotating within the lumen when the trough is in the lumen.

4. The material delivery apparatus of claim 3, wherein the locking element comprises a flange at a proximal end of the trough that is received within a recess in a grip of the sheath.

5. The material delivery apparatus of claim 1, wherein the sheath, trough, stylet, or any combination of the sheath, trough, and stylet, includes a radio-opaque marking.

6. The material delivery apparatus of claim 1, wherein the stylet further comprises a handle at a proximal end of the stylet.

7. The material delivery apparatus of claim 1, wherein the sheath further comprises a grip at a proximal end of the sheath.

8. The material delivery apparatus of claim 1, wherein the sheath is shaped such that the trough passes into the lumen and the material receptacle in only one orientation.

9. The material delivery apparatus of claim 1, further comprising a handle configured to advance the stylet a predetermined distance, such that a predetermined amount of material is dispensed.

10. A material delivery apparatus for delivering bone graft material to a surgical site, the material delivery apparatus comprising:
a sheath that defines a lumen therethrough;
a trough configured to slidably pass into the lumen and having an inner surface that defines a material receptacle; and
a stylet configured to slidably pass into the material receptacle,
the stylet having a cross-sectional shape that complements the cross-sectional shape of a void defined between the inner surface of the trough and the sheath when the trough is within the lumen of the sheath.

11. The material delivery apparatus of claim 10, wherein the trough is configured to receive the bone graft material in the material receptacle.

12. The material delivery apparatus of claim 10, wherein the stylet is configured to displace a portion of the bone graft material from the material receptacle as the stylet slides into the material receptacle.

13. The material delivery apparatus of claim 10, wherein the stylet is shaped to be inserted into the lumen in only one orientation.

14. The material delivery apparatus of claim 10, wherein the trough further includes a locking element configured to releasably secure the trough to the sheath when the trough is in the lumen, such that when the trough is in the lumen, the trough is prevented from rotating within the lumen.

15. The material delivery apparatus of claim 14, wherein the locking element comprises a flange at a proximal end of the trough that is received within a recess in a grip of the sheath.

16. The material delivery apparatus of claim 10, wherein the sheath, trough, stylet, or any combination of the sheath, trough, and stylet, includes a radio-opaque marking.

17. The material delivery apparatus of claim 10, further comprising a packing tool, the packing tool having a scraper configured to engage with the bone graft material and move the bone graft material into the material receptacle.

18. The material delivery apparatus of claim 17, wherein the packing tool further comprises a tamping head configured to be applied to the bone graft material in the material receptacle, and wherein the tamping head has a curved shape.

19. A method of loading bone graft material into a material delivery apparatus having a sheath, a trough, and a keyed stylet, the method comprising:
introducing the bone graft material into a material receptacle defined by an inner surface of the trough;
slidably positioning the trough into a lumen defined by the sheath; and
sliding the keyed stylet into an entirety of a void defined by the sheath and the trough.

20. The method of claim 19, wherein sliding the keyed stylet comprises displacing bone graft material out of the lumen of the sheath.

21-34. (canceled)
